(19) United States
(12) Patent Application Publication
(10) Pub. No.: US 2024/0398789 A1
(43) Pub. Date: Dec. 5, 2024

(54) PHARMACEUTICAL COMPOSITION FOR ENHANCING ANTI-TUMOR EFFECT OF EZH2 INHIBITOR AND USE THEREOF

(71) Applicant: PEKING UNIVERSITY THIRD HOSPITAL, BEIJING (CN)

(72) Inventors: Lixiang XUE, BEIJING (CN); Yan WANG, BEIJING (CN); Tengrui ZHANG, BEIJING (CN); Zhengyang GUO, BEIJING (CN); Yueqing GONG, BEIJING (CN); Qianqian YIN, BEIJING (CN); Yan SUN, BEIJING (CN); Hui MENG, BEIJING (CN); Xiaojuan MA, BEIJING (CN)

(73) Assignee: PEKING UNIVERSITY THIRD HOSPITAL, BEIJING (CN)

(21) Appl. No.: 18/701,160

(22) PCT Filed: Oct. 13, 2022
(86) PCT No.: PCT/CN2022/125101

§ 371 Date: Apr. 12, 2024

(30) Foreign Application Priority Data
Oct. 15, 2021 (CN) 202111201508.4
Oct. 9, 2022 (CN) 202211226629.9

(51) Int. Cl. A61K 31/496; A61K 31/216; A61P 35/00

(52) U.S. Cl. CPC A61K 31/496; A61K 31/216; A61P 35/00

(57) ABSTRACT

The present invention belongs to the field of medicine. Provided is a pharmaceutical composition for enhancing the anti-tumor effect of an EZH2 inhibitor. The pharmaceutical composition comprises an EZH2 inhibitor and a PPARαagonist, wherein the PPARα agonist enhances the anti-solid tumor effect of the EZH2 inhibitor. Further provided in the present invention are a related preparation of the pharmaceutical composition and the use thereof in the preparation of an anti-tumor drug. Experimental results show that when the EZH2 inhibitor GSK126 and the PPARα agonist fenofibrate are used in combination to treat B16F10 cells, SMMC7721 cells and GL261 cells, it is found that the use thereof in combination with fenofibrate can significantly enhance the inhibitory effect of GSK126 on cell proliferation. The pharmaceutical composition of the present invention provides a new thought for the clinical treatment of solid tumors by means of safely, effectively, conveniently and economically using the EZH2 inhibitor, has good clinical application prospects, and provides evidence for a new use of the conventional drug fenofibrate.

### RELATED APPLICATIONS

This application claims the benefit of Chinese Application No. 2021112015084 filed on Oct. 15, 2021 and claims the benefit of Chinese Application No. 2022112266299 filed on Oct. 9, 2022. The application Ser. No. 20/211,12015084 and 2022112266299 are hereby incorporated by reference in their entirety.

### Field

The invention belongs to the field of medicines, and particularly relates to a pharmaceutical composition for enhancing the anti-tumor effect of an EZH2 inhibitor, and use of the pharmaceutical composition in the preparation of anti-tumor drugs.

### Background

Enhancer of zeste homolog 2 (EZH2) is the catalytic subunit of the polycomb repressive complex 2 (PRC2) and functions as a histone methyltransferase. The abnormality of EZH2 appears in many diseases (such as tumors), and several evidences show that EZH2 is related to the occurrence and progression of many cancers, as well as their poor prognosis. Overexpression of EZH2 is mainly occurred in solid tumors, including prostate cancer, breast cancer, bladder cancer, endometrial cancer, and melanoma, etc. High level of EZH2 expression is often associated with high aggressiveness, tumor progression, and poor clinical outcome and prognosis in these types of tumors.

Unlike normal cells, tumor cells directly or indirectly regulate cellular metabolic reprogramming through oncogenic mutations to meet the demands for their survival and proliferation. Epigenetic mechanisms can regulate the expression of genes involved in metabolism, thereby changing the metabolic characteristics of cells. As a key regulator of histone modification, EZH2 is involved in regulating a variety of metabolic activities of tumor cells, thus affecting the progression of cancer.

EZH2 can also promote lipid synthesis in tumor cells. In glioma cells containing telomerase reverse transcriptase (TERT) mutations, there was a positive correlation between TERT and EZH2 levels. TERT and EZH2 synergistically activate peroxisome proliferator-activated receptor gamma coactivator 1-α (PGC-1α), while the expression of fatty acid synthase (FASN) depends on PGC-1α, so that EZH2 promotes fatty acid synthesis and accumulation through TERT-EZH2 network. It has been reported that high levels of fatty acids in tumor cells can promote tumorigenesis and drug resistance by negatively regulating the DNA damage repair (DDR) pathway. In contrast, DZNep, an EZH2 inhibitor, induces lipid accumulation in nonalcoholic fatty liver cells and certain cancer cell lines, such as breast cancer. In order to clarify this difference, the role of EZH2 in adipocyte differentiation and lipid metabolism has been studied by using primary human or mouse preadipocytes and mice with specific knockout of EZH2 in adipocytes. It has been found that inhibition of EZH2 or deletion of its gene has promoted the up-regulation of Apolipoprotein E (ApoE) gene expression, which is accompanied by lipoprotein-dependent lipid absorption and ultimately leads to intracellular lipid accumulation. However, it does not affect the expression of adipocyte marker genes and adipocyte differentiation. This is contrary to the conclusion that EZH2 promotes adipogenesis and adipocyte differentiation of mouse adipose progenitor cells in previous studies. Therefore, the role of EZH2 in the regulation of lipid metabolism in tumor cells is not very clear. It is still demanded for further investigation on how EZH2 affects fatty acids, triglycerides, ketone bodies and other lipid metabolites, and what role these metabolites play in the progression of tumors.

Many efficient and selective EZH2 catalytic inhibitors have been obtained through high-throughput screening, such as EPZ005687, EI1, GSK343, GSK126 and so on, almost all of which have 2-pyridone group in their structures. A number of EZH2 inhibitors have been developed at present as potential anti-cancer agents. Among others, CPI1205(Lirametostat) has been tested in clinical trials, and EPZ-6438 (Tazemetostat) was approved by the FDA for the treatment of epithelioid sarcoma in 2020. However, EZH2 inhibitors are not effective in EZH2-overexpressing solid tumors, such as glioma and melanoma, which can escape the anti-tumor effect of EZH2 inhibitors through simultaneous mutations in the Ras pathway and SWI/SNF. Therefore, it has been attempted to improve the efficacy of EZH2 inhibitors by employing a therapeutic strategy combining multiple drugs or multiple anti-tumor therapies (see, e.g., Zhang Tengrui, et al., Symphony of epigenetic and metabolic regulation-interaction between the histone methyltransferase EZH2 and metabolism of tumor, Clinical Epigenetics, 2020, 12:72).

In addition, the inventor's previous research results suggested that epigenetic regulation and metabolic changes mediated by EZH2 showed a synergistic effect in cancer cells. The inventor has preliminarily found that the poor therapeutic effect of the EZH2 inhibitor may be caused by lipid metabolism disorder. In addition, several typical lipid-lowering drugs, especially fibrates and statins, have been found to have anti-cancer effects in many studies. Fenofibrate (FF) has anti-cancer activities against breast cancer, oral tumors, melanoma, lung cancer, glioblastoma and liver cancer. The related effects are involved in promoting cell apoptosis, arresting cell cycle, inhibiting invasion and migration, etc.

In view of the above research background, the inventor speculates that the combination of lipid metabolism drugs may play an important role in the combination therapy of some anti-tumor drugs, which may provide a new idea for addressing the poor therapeutic effect of EZH2 inhibitors.

### SUMMARY

Aiming at the poor activity of an EZH2 inhibitor on a solid tumor, the objective of the present invention is to provide a pharmaceutical composition comprising an EZH2 inhibitor and a PPARα agonist for enhancing the anti-tumor effect of the EZH2 inhibitor, and to provide a new thought for the clinical treatment of solid tumors by means of safely, effectively, conveniently and economically using the EZH2 inhibitor.

Specifically, the present invention is implemented by the following technical solutions:

In a first aspect, the present invention provides a pharmaceutical composition for enhancing the anti-tumor effect of an EZH2 inhibitor, wherein the pharmaceutical composition comprises an EZH2 inhibitor and a PPARα agonist, and wherein the PPARα agonist enhances the anti-solid tumor effect of the EZH2 inhibitor.

Alternatively, in the above pharmaceutical composition, the mass ratio of the EZH2 inhibitor to the PPARα agonist is from 1:1 to 1:10. Substantially, the dosage ratio of the EZH2 inhibitor to the PPARα agonist in the pharmaceutical composition may be determined by a clinician based on clinical experience according to the type of cancer suffered by the patient.

Preferably, the mass ratio of the EZH2 inhibitor to the PPARα agonist is selected from 1:1, 1:2, 1:3, 1:4, 1:5, 1:6, 1:7, 1:8, 1:9, or 1:10.

Alternatively, in the above pharmaceutical composition, the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer.

Preferably, the solid tumor is selected from breast cancer, melanoma, lung cancer, colon cancer, liver cancer, gastric cancer, or brain cancer.

More preferably, the brain cancer is selected from meningioma, glioma (e.g., astrocytoma, oligodendroglioma), or medulloblastoma.

Alternatively, in the above pharmaceutical composition, the EZH2 inhibitor is selected from Tazemetostat (EPZ-6438), GSK126, Lirametostat (CPI-1205), SHR2554 or PF-06821497; the PPARα agonist is selected from fenofibrate, clofibrate, bezafibrate, clinofibrate, ciprofibrate, etofibrate, gemfibrozil, WY-14643 (pirinixic acid), GW-7647(2-(4-(2-(1-1-cyclohexylbutyl)-3-cyclohexylureido)ethyl)phenylthio)-2-methylpro pionic acid) or pemafibrate ((R)-2-{3-{[N-(benzoxazol-2-yl)-N-3-(4-methoxyphenoxy) propyl]aminomethyl} phenoxy} butyric acid).

Alternatively, in the above pharmaceutical composition, preferably, the EZH2 inhibitor is GSK126, and the PPARα agonist is fenofibrate.

In a second aspect, the present invention provides a pharmaceutical formulation for enhancing the anti-tumor effect of an EZH2 inhibitor, wherein the pharmaceutical preparation is prepared from a therapeutically effective amount of the pharmaceutical composition of the first aspect above and a pharmaceutically acceptable carrier.

Alternatively, in the above pharmaceutical preparation, the pharmaceutical preparation is an oral preparation.

Preferably, the oral preparation is an oral liquid, a tablet, a powder, a capsule or a granule.

In a third aspect, the present invention provides the use of the pharmaceutical composition of the first aspect or the pharmaceutical preparation of the second aspect in the preparation of an anti-tumor drug.

Alternatively, in the above use, the tumor is a solid tumor.

The solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer, and preferably, the solid tumor is selected from breast cancer, melanoma, lung cancer, colon cancer, liver cancer, gastric cancer, or brain cancer.

More preferably, the brain cancer is selected from meningioma, glioma (e.g., astrocytoma, oligodendroglioma), or medulloblastoma.

In a fourth aspect, the present invention provides the use of a PPARα agonist in the preparation of a drug for enhancing the efficacy of an EZH2 inhibitor against a solid tumor.

The solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer.

Preferably, the solid tumor is selected from breast cancer, melanoma, lung cancer, colon cancer, liver cancer, gastric cancer, or brain cancer.

More preferably, the brain cancer is selected from meningioma, glioma (e.g., astrocytoma, oligodendroglioma), or medulloblastoma.

The EZH2 inhibitor is selected from Tazemetostat (EPZ-6438), GSK126, Lirametostat (CPI-1205), SHR2554 or PF-06821497.

The PPARα agonist is selected from fenofibrate, clofibrate, bezafibrate, clinofibrate, ciprofibrate, etofibrate, gemfibrozil, WY-14643 (pirinixic acid), GW-7647(2-(4-(2-(1-1-cyclohexylbutyl)-3-cyclohexylureido)ethyl)phenylthio)-2-methylpro pionic acid) or pemafibrate ((R)-2-{3-{[N-(benzoxazol-2-yl)-N-3-(4-methoxyphenoxy)propyl]aminomethyl}phenoxy} butyric acid).

It should be understood that within the scope of the present invention, the various technical features of the present invention described above and those specifically described below (e.g., examples) may be combined with each other to form new or preferred embodiments. It will not be repeated for the sake of simplicity.

As compared with that prior art, the invention has the following beneficial effects:

The inventor discovers for the first time that the efficacy of the EZH2 inhibitor on solid tumors can be significantly enhanced by the combination of the EZH2 inhibitor and the PPARα agonist, and on such a basis, a pharmaceutical composition comprising the EZH2 inhibitor and the PPARα agonist is provided for enhancing the anti-tumor effect of the EZH2 inhibitor. The present invention provides a new thought for the clinical treatment of solid tumors by means of safely, effectively, conveniently and economically using the EZH2 inhibitor, and has good clinical application prospects.

### BRIEF DESCRIPTION OF THE DRAWINGS

FIG. 1: Hematological tumors are more sensitive to GSK126 than solid tumors. (A) Proliferation of B16F10, Huh7 and SMMC7721 cells in GSK126 (10-20 μM) treatment was monitored by Incucyte S3 with 6 replicates per group; (B) Viabilities of Daudi and THP-1cells after GSK126 (200 nM to 24 μM) treatment for 24 h and 48 h were measured by CCK-8 assay with 5 replicates per group; (C) Western blot (WB) analysis on B16F10 cells after 48 h without or with GSK126 treatment (6 μM, and 12 μM), with H3 used as an internal reference for histone. 5 to 6 replicates were established for each group, with *P<0.05, **P<0.01, and ***P<0.001, and ns for no statistical difference.

FIG. 2: Melanoma cells B16F10 are less sensitive to GSK126. (A) Cell scratch assay, showing the effect of GSK126 (10 μM) treatment on the migration capability of B16F10 cells, with 3 replicates per group; (B-C) the effect of GSK126 (10, 13, and 15 μM) on the apoptosis rate of B16F10 cells, as examined by flow cytometry (C) and Annexin V staining assay. 3 replicates were established for each group, with *P<0.05, **P<0.01, and ***P<0.001, and ns for no statistical difference.

FIG. 3: GSK126 results in significant elevated levels of 6 fatty acids in melanoma B16F10 cells.

FIG. 4: Lipid accumulation in mice after GSK126 treatment. (A) H & E and oil red O staining of the liver tissue sections of the tumor-bearing mice in the control group and the GSK126 treatment group, in which the statistical results of negative or positive staining area were shown on the right (3 replicates per group); (B) TG level in the serum of the tumor-bearing mice in the control group and the GSK126 treatment group (5 replicates per group); (C-D) mRNA (C) and protein (D) levels of the genes related to fatty acid anabolism in the GSK126-treated HCC cells, as detected by RT-qPCR and WB (3 replicates per group). 3 to 5 replicates were established for each group, with *P<0.05, **P<0.01.

FIG. 5: Lipid metabolism-regulating drugs can inhibit the proliferation of tumor cells. (A) The mRNA levels of ACLY and FASN were up-regulated in the tissues of patients with liver cancer (GSE14520) and melanoma (GSE3189) based on ONCOMINE database analysis; (B) Kaplan-Meier survival curves for the patients in the TCGA LIHC (liver cancer) and GSE8401 (melanoma) datasets. The overall survival was significantly reduced in the HCC patients with higher ACLY expression and the melanoma patients with higher FASN expression. P=0.0108, P=0.0385, Kaplan-Meier survival analysis; (C) effect of telmisartan at different concentrations on the proliferation of U87 cells (left) and U251 cells (right) (3 replicates per group); (D) effect of fenofibrate at different concentrations on the proliferation of B16F10 cells (left) and 4T1 cells (right) (3 replicates per group); (E) FACS quantitative analysis of the percentage of cells positive for EdU (FITC+) staining 48 h after the telmisartan treatment on U87 and U251 cells. Single cells were first gated, followed by FITC+cells. The percentage of FITC+ cells was listed above the corresponding cell population. 3 replicates were established for each group, with *P<0.05, **P<0.01, ***P<0.001, and ****P<0.0001, and ns for no statistical difference.

FIG. 6: Lipid-modulating drugs can enhance the inhibitory effect of GSK126 on cancer cells. (A) The proliferation of B16F10 and SMMC7712 cells was monitored by Incucyte S3 in four groups, i.e., the control group, the fenofibrate group (25 μM, FF50), the GSK126 group (10 μM, G10), and the combination group (fenofibrate 25 μM and GSK126 10 μM, GSK126+FF) (5 replicates per group); (B) mRNA levels of the genes related to fatty acid synthesis in the B16F10 cells and the SMMC7712 cells treated under the same conditions as (A) were determined by qRT-PCR (3 replicates per group). 3 to 5 replicates were established for each group, with *P<0.05, **P<0.01, and ***P<0.001.

FIG. 7: Lipid-modulating drugs can enhance the inhibitory effect of GSK126 on brain cancer cells. The proliferation of GL261 cells was monitored by Incucyte S3 in four groups, i.e., the control group, the fenofibrate group (10 μM), the GSK126 group (20 μM, and 40 μM), and the combination group (fenofibrate 10 μM and GSK126 20 μμM, GSK126+FF). 3 to 6 replicates were established for each group, with *P<0.05, **P<0.01, and ***P<0.001, and ns for no statistical difference.

### DETAILED DESCRIPTION

The inventor discovers for the first time that the efficacy of the EZH2 inhibitor for solid tumors can be significantly enhanced by the combination of the EZH2 inhibitor with the PPARα agonist through a large amount of screening experiments during a thoroughly research on the regulation mechanism of EZH2 on lipid metabolism in tumor cells and the anti-tumor mechanism of the EZH2 inhibitor. On such a basis, the present invention has been accomplished.

As used herein, the EZH2 inhibitor and the PPARα agonist in the pharmaceutical composition of the present invention may be administered in the same pharmaceutical formulation or in different pharmaceutical formulations. The dosage forms of the EZH2inhibitor and the PPARα agonist may be the same or different when administrated in different pharmaceutical formulations. Moreover, the EZH2 inhibitor and the PPARα agonist may be administered simultaneously or sequentially.

As used herein, in the present invention, the PPARα agonist refers to a general term for a compound that activates PPARα receptor, which is involved in fat oxidation and the like in a peroxisome proliferator-responsive receptor (PPAR) that is one of the intranuclear receptors. Specific examples include fibrates such as fenofibrate, clofibrate, bezafibrate, clinofibrate, ciprofibrate, etofibrate, and gemfibrozil, etc.; WY 14643 (pirinixic acid); GW-7647(2-(4-(2-(1-1-cyclohexylbutyl)-3-cyclohexylureido) ethyl)phenylthio)-2-methylpropionic acid); pemafibrate ((R)-2-{3-{[N-(benzoxazol-2-yl)-N-3-(4-methoxyphenoxy)propyl]aminomethyl}phenoxy} butyric acid) and the like.

Non-limiting examples of tumors treatable with the pharmaceutical compositions of the present invention may include, but are not limited to, biliary tract cancer (e.g., cholangiocarcinoma), bladder cancer, breast cancer (e.g., breast adenocarcinoma, papillary breast cancer, breast cancer, medullary breast cancer, triple negative breast cancer, HER2 negative breast cancer, HER2 positive breast cancer, male breast cancer, advanced metastatic breast cancer, progesterone receptor negative breast cancer, progesterone receptor positive breast cancer, relapsed breast cancer), brain cancer (e.g., meningioma; gliomas such as astrocytomas, oligodendrogliomas; medulloblastoma), bronchial cancer, cervical cancer (e.g., cervical adenocarcinoma), choriocarcinoma, colorectal cancer (e.g., colon cancer, rectal cancer, colorectal adenocarcinoma), epithelial cancer, endometrial cancer (e.g., uterine cancer, uterine sarcoma), esophageal cancer (e.g., esophageal adenocarcinoma, Barrett's adenocarcinoma), Ewing's sarcoma, ocular cancer (e.g., intraocular melanoma, retinoblastoma), gallbladder cancer, gastric cancer (e.g., gastric adenocarcinoma), gastrointestinal stromal tumor (GIST), glioblastoma multiforme, head and neck cancer (e.g., head and neck squamous cell carcinoma, oral cancer (e.g., oral squamous cell carcinoma (OSCC)), throat cancer (e.g., laryngeal cancer, pharyngeal cancer, rhinopharynx cancer, oropharyngeal cancer)), kidney cancer (e.g., nephroblastoma, also known as Wilms' tumor, renal cell carcinoma), liver cancer (e.g., hepatocellular carcinoma (HCC), malignant liver cancer), lung cancer (e.g., bronchial carcinoma, small cell lung carcinoma (SCLC), non-small cell lung carcinoma (NSCLC), lung adenocarcinoma), leiomyosarcoma (LMS), myelodysplastic syndrome (MDS), mesothelioma, neuroendocrine carcinoma (e.g., gastroenteropancreatic neuroendocrine tumor (GEP-NET), carcinoid tumor), osteosarcoma, ovarian carcinoma (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), pancreatic islet cell tumor), penile cancer (e.g., Paget's disease of the penis and scrotum), prostate cancer (e.g., prostatic adenocarcinoma), rectal cancer, rhabdomyosarcoma, skin cancer (e.g., squamous cell carcinoma (SCC), corneal acanthoma (KA), melanoma, basal cell carcinoma (BCC)), small intestine carcinoma (e.g., appendiceal carcinoma), soft tissue sarcoma (e.g., malignant fibrous histiocytoma (MFH), liposarcoma, chondrosarcoma, fibrosarcoma), sebaceous gland carcinoma, sweat gland carcinoma, synovioma, testicular carcinoma (e.g., seminoma, testicular embryonal carcinoma), thyroid carcinoma (e.g., thyroid papillary carcinoma, papillary thyroid carcinoma (PTC), medullary thyroid carcinoma), urethral carcinoma, vaginal carcinoma, and vulvar carcinoma (e.g., vulvar Paget's disease).

As used herein, the dosage forms of the pharmaceutical formulation of the present invention is selected from the group consisting of a tablet, a capsule, a granule, oral liquid or inhalant. Preferably, the dosage form of the present invention is a tablet or a capsule.

As used herein, the “pharmaceutically acceptable carrier” of the present invention refers to a conventional pharmaceutical carrier in the field of pharmaceutical formulation, which is selected from one or more of a filler, a binder, a disintegrant, a lubricant, a suspending agent, a wetting agent, a pigment, a flavoring agent, a solvent, and a surfactant.

The filler of the invention includes, but not limited to, starch, microcrystalline cellulose, sucrose, dextrin, lactose, powdered sugar, glucose and the like; the lubricant includes, but not limited to, magnesium stearate, stearic acid, sodium chloride, sodium oleate, sodium lauryl sulfate, poloxamer and the like; the binder includes, but is not limited to, water, ethanol, starch slurry, syrup, hydroxypropyl methyl cellulose, sodium carboxymethyl cellulose, sodium alginate, polyvinylpyrrolidone, and the like; the disintegrant includes, but is not limited to, starch effervescent mixture, namely sodium bicarbonate and citric acid, tartaric acid, low-substituted hydroxypropyl cellulose, and the like; the suspending agent includes, but is not limited to, polysaccharides such as gum acacia, agar, alginic acid, cellulose ether, carboxymethyl chitosan, and the like; and the solvent includes, but are not limited to, water, equilibrated salt solutions, and the like.

Preferably, the drug of the present invention can be prepared into various solid oral formulations, liquid oral formulations, and the like. The pharmaceutically acceptable oral solid preparations include ordinary tablets, dispersible tablets, enteric coated tablets, granules, capsules, dropping pills, pulvis, and the like. The oral liquid preparations include oral liquid, emulsion, and the like. Alternatively, the drug of the present invention may be prepared into a topical dosage form, such as an inhalant, and the like.

The various dosage forms described above can be prepared according to the conventional techniques in the field of pharmaceutical formulation.

In the pharmaceutical composition, pharmaceutical preparation and medical use described above, the administration time, number, and frequency of “an EZH2 inhibitor” and “PPARα agonist”, and the like are determined according to the specific diagnosis of the diseases, which is well within the scope of the skills of those skilled in the art. For example, when a treatment regimen for mice or rats is applied to a human body, the effective dose of all drugs to the human body can be calculated based on the effective dose of the drugs to the mice or rats, which can also be easily realized by a person of ordinary skill in the art.

The invention is further described below with reference to specific examples. It should be understood that the specific examples described herein are merely illustrative of the invention, and are not intended to limit the scope of the invention.

If no specific technology or conditions are specified in the examples, the technology or conditions described in the literature in this field or in the product specification shall be followed. If no manufacturer is indicated for the reagent or instrument used, it is a conventional product that can be purchased through regular channels.

The experimental methods in the following examples are all conventional methods, unless otherwise specified. The test materials used in the following examples are commercially available, unless otherwise specified.

The percentages and parts referred to in the present invention are weight percentages and weight parts, unless otherwise stated.

### EXAMPLES:

1. Many solid tumor cell lines have low sensitivity to EZH2 inhibitor GSK126.

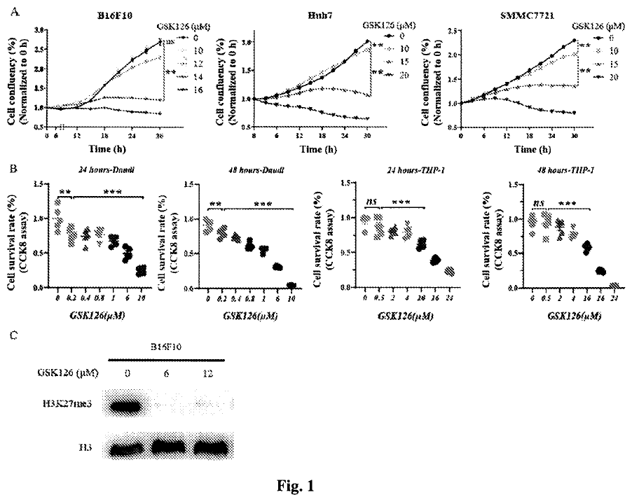

In order to accurately assess the responsiveness of solid tumor cells to EZH2 inhibitors, the effects of GSK126 at different concentrations on the proliferation of melanoma B16F10 cells and hepatoma Huh7 and SMMC-7721 cells were initially detected by IncuCyte S3® Live-Cell Analysis System. The results showed that GSK126 inhibited cell proliferation in a dose-dependent manner, whereas GSK126 at a higher concentration of 10 μM was less effective (FIG. 1A).

Under the same conditions, the results of CCK-8 cell proliferation assay showed that the proliferation of hematological tumor cells Daudi and THP-1 was significantly inhibited after treated with 10 μM GSK126 for 24 h and 48 h. In particular, Burkitt's lymphoma cells (Daudi) were already very sensitive to 1 μM GSK126 (FIG. 1B). As consistent with other studies, we have found that solid tumor cell lines are generally insensitive to EZH2 inhibitors, whereas many studies have shown that the IC50 (half-maximal inhibitory concentration) of most hematologic cancer cell lines treated with GSK126 is lower than 1 μM.

Moreover, it was observed that the H3K27me3 level in B16F10 cells was significantly down-regulated by GSK126 at 6 μM (FIG. 1C), but the proliferation of cancer cells could not be effectively inhibited by GSK126 at 10 μM (FIG. 1A). This indicates that GSK126 can not exert its anti-tumor effect, when it has been able to effectively inhibit the histone methyltransferase activity of EZH2.

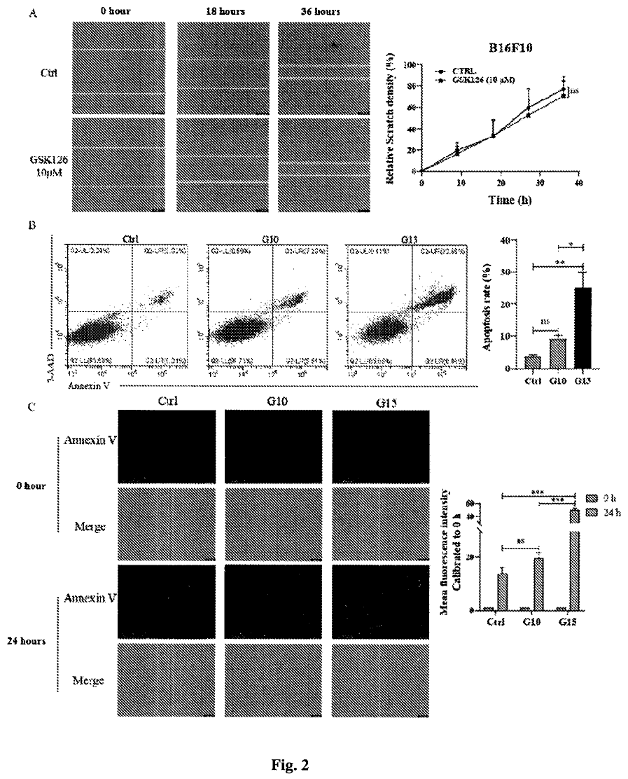

Next, to further validate the effect of GSK126 at 10 μM on melanoma cells B16F10,scratch assay was conducted. Similar to the cell proliferation assay, after 36 h of culture, GSK126 did not effectively inhibit cell migration at 10 μM as compared with the control group (FIG. 2A). In addition, to assess the anti-survival effect of GSK126, B16F10 cells were treated by GSK126 at different concentrations for different periods, and the apoptosis rate of the cells was analyzed by flow cytometry (FIG. 2B) and live-cell fluorescence imaging (FIG. 2C). The results indicate that GSK126 at 10 μM has a limited capability to induce apoptosis in B16F10 cells. Accordingly, the proliferation of solid tumor cells (B16F10, Huh7 and SMMC7721) and the migration and survival of B16F10 cells were not significantly inhibited after treated by GSK126 at higher concentration (10 μM).

2. GSK126 up-regulates the fatty acid levels in melanoma B16F10 cells

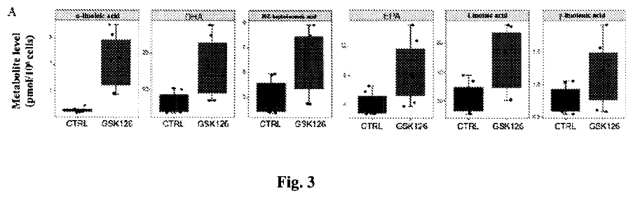

Among the metabolites with significant changes, the levels of many fatty acids increased in the GSK126 group. There are five polyunsaturated fatty acids, including α-linolenic acid, DHA, EPA, linoleic acid, and γ-linolenic acid) and one monounsaturated fatty acid, i.e., 10Z-heptadienoic acid (FIG. 3).

This indicates that GSK126 can improve the fatty acid abundance. These fatty acids can be used as substrates for lipid synthesis, form structures of plasma membrane, but also inhibit cell growth and induce apoptosis when excessively accumulated. De novo synthesis of fatty acids can provide raw materials for the biofilm structure, energy production and protein modification of cancer cells. Moreover, high LD and cholesteryl ester content in tumors are correlated with cancer invasiveness. This suggests that lipid accumulation may be responsible for the poor anti-tumor efficacy of GSK126.

3. Regulation of lipid metabolism can enhance the inhibitory effect of GSK126 on cancer cells.

3.1 GSK126 treatment leads to in vivo accumulation of lipids in mice

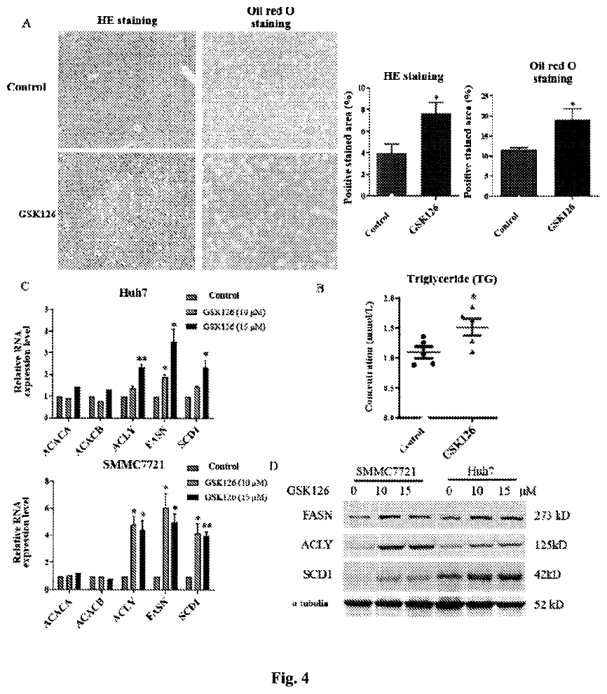

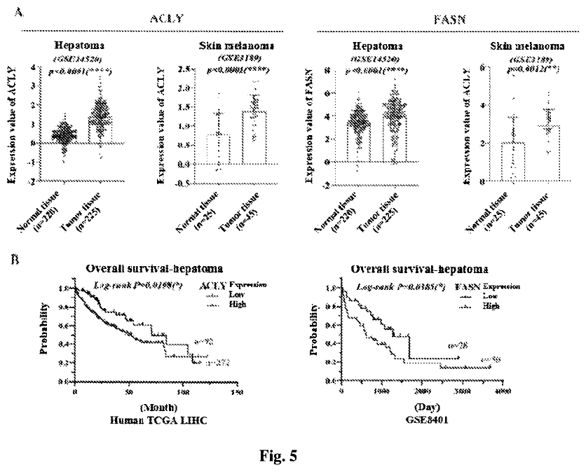

Since a plurality of fatty acids were up-regulated in the B16F10 cells treated with GSK126, alterations in lipid metabolism in tumor-bearing mice were explored. C57BL/6 mice (6-8 weeks old) were subcutaneously implanted with B16F10 cells and then treated with GSK126. H & E and oil red O staining of liver tissue sections from mice showed a significant increase in fat vacuoles and lipid deposition in the GSK126 group (FIG. 4A). The levels of triglycerides (TG) were also significantly elevated in the blood of mice (FIG. 4B). These results suggest that GSK126 can regulate lipid metabolism in the liver and throughout the body. Therefore, the expression of fatty acid synthesis-related genes in GSK126-treated hepatocellular carcinoma (HCC) cell lines SMMC7721 and Huh7 was further detected. As shown in FIG. 4C-D, mRNA and protein levels of ACLY, FASN, and SCD were significantly elevated after GSK126 treatment, which was consistent with the RNA-seq data.

3.2 Lipid metabolism-regulating drugs can inhibit the proliferation of tumor cells

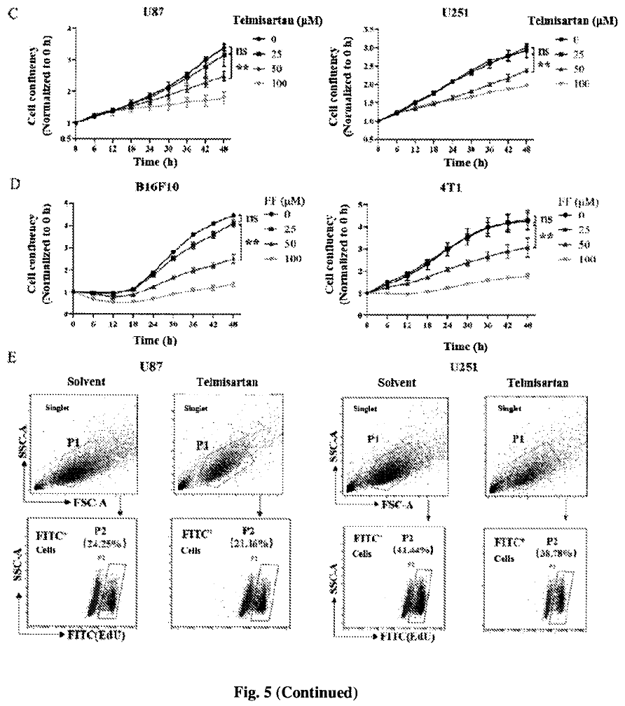

Previous studies have shown that these metabolic enzymes that promote adipogenesis show relatively strong activity and high expression in tumor cells. According to the analysis in Oncomine database (TCGA_LIHC and GSE8401 dataset), the expression of FASN and ACLY was found to be up-regulated in liver cancer and melanoma, respectively (FIG. 5A). Meanwhile, high expression of ACLY and FASN was associated with poor prognosis in patients with liver cancer and melanoma, respectively (FIG. 5B). The above results indicate that increased lipid synthesis play an important role in the development and progression of cancer.

PPAR family is a group of key factors for regulating glucose and lipid homeostasis, and PPARα agonist fenofibrate and PPARγ agonist telmisartan have been found to have certain anti-tumor efficacy in preclinical studies. Therefore, telmisartan and fenofibrate were selected to explore whether lipid metabolism-regulating drugs have anti-tumor effects. Telmisartan was found to inhibit the proliferation of human glioma cells U87 and U251 in a dose-dependent manner (FIG. 5C), and fenofibrate was also able to inhibit the proliferation of melanoma cells B16 and breast cancer cells 4T1 at certain concentrations (FIG. 5D), as detected by Incucyte cell real-time monitoring system. The effect of telmisartan (50 μM) on cell proliferation was further verified by EdU staining. The results of flow cytometry showed that the percentage of EdU+ U87 cells decreased from 24.25% to 21.16% and the percentage of EdU+ U251 cells decreased from 41.44% to 38.78% after telmisartan treatment (FIG. 5E).

3.3 Regulation of lipid metabolism can enhance the inhibitory effect of GSK126 on cancer cells

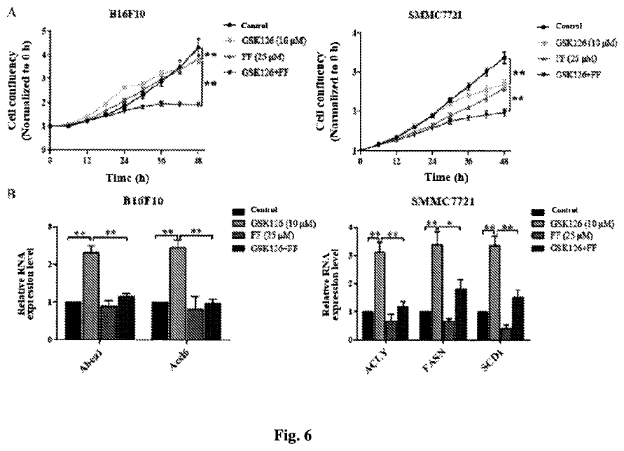

It can be seen from Section 3.2 that lipid metabolism-regulating drugs can indeed inhibit cell proliferation. At the same time, an increase in lipid synthesis by the cells was observed after GSK126 treatment, which may explain why the sensitivity of various tumor cells was not good after GSK126 treatment at 10 μM, so that it was further explored whether increased lipolysis could enhance the inhibitory effect of GSK126 on cancer cells. Previous studies have shown that there is an upstream and downstream regulatory relationship between EZH2 and PPARα, and both EZH2 and PPARα play a role in the regulation of lipid metabolism. Therefore, the combination of GSK126 with the PPARα agonist fenofibrate was used to treat B16F10 and SMMC7721 cells, and it was found that this combination did significantly enhance the inhibitory effect of GSK126 on cell proliferation (FIG. 6A). At the same time, it was also demonstrated that the combination of drugs could down-regulate the elevated level of lipid metabolism caused by GSK126treatment. It was found that the expression of lipid metabolism genes ATP-binding cassette subfamily A, member 1 (Abca1) and Acsl6 was significantly reduced in B16F10 cells in the combination group as compared to the GSK126 group. Meanwhile, in SMMC7712 cells, fatty acid synthesis genes (including ACLY, FASN, and SCD1) were significantly decreased in the combination group (FIG. 6B).

3.4 Regulation of lipid metabolism may enhance the inhibitory effect of GSK126 on brain cancer cell lines

3.4.1 Experiment methods

IncuCyte S3® Live-Cell Analysis System was used to monitor and analyze the growth of mouse glioma GL261 cells.
(1) GL-261 cells in logarithmic growth phase were prepared into a single cell suspension, and the cells were counted;
(2) GL261 cells were added into a 96-well plate at a density of 8x10⁴ cells/mL, with 4-6 replicate wells per each group;
(3) The 96-well plate was then placed in an incubator for another 12-24 hours, and the plate was refreshed with a culture medium containing the drug at the cell confluence of about 20-30%. GSK126 and fenofibrate (FF) were serially diluted using the medium;
(4) The 96-well plate was then placed in the incubator suitable for the instrument, and detected with IncuCyte S3® Live-Cell Analysis System. Cell growth images of 4 different areas in each well were taken at an interval of 4 h using a 10× objective lens.
(5) The plate was real-time monitored for 48 h, and the cell confluence was determined by defining the cell edge using IncuCyte S3 image analysis software and normalized to the cell confluence at 0 h. The percentage was calculated and the proliferation curve was plotted to evaluate the cell proliferation;
(6) At the end of the culture, it was also detected by the MTT method using a spectrophotometer.

3.4.2 Experiment results

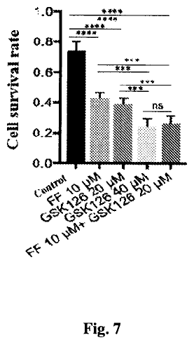

The experiment results are shown in FIG. 7. Among others, the control group was only given the solvent DMSO. The experiment results showed that the inhibitory effect of GSK126 at 40 μM on mouse brain glioma cell line (GL261) was the same as that of the combination of GSK126 at 20 μM with fenofibrate (FF) at 10 μM. This effect can not achieved by 20 μM GSK126 alone. That is to say, the combination of low-dose of GSK and fenofibrate (FF) can achieve the effect that high-dose of GSK126 can, indicating that in brain glioma, the combination of both drugs has better effect.

3.5 Anti-tumor effect of the combination of EZH2 inhibitor with PPARα agonist in a common tumor-bearing mouse model

3.5.1 Establishment of tumor-bearing mouse model

For the mouse colon tumor model:
(1) A single cell suspension of mouse colon cancer cells (MC38) in logarithmic growth phase was prepared. The cell concentration was adjusted to 1.0x10⁷ cells/mL using sterile PBS as solvent;
(2) C57BL/6 female mice, 6-8 weeks old, weighing 18-20 g, were anesthetized with sodium phenobarbital (40 mg/kg) in an superclean bench and shaved to facilitate tumor-bearing and tumor measurement;
(3) MC38 cells (0.1 mL of single cell suspension) were subcutaneously implanted into the upper right area of the back of the mouse with a syringe, and an obvious spherical bulge was formed at the injection site;
(4) The mice were returned to the rearing cage and subsequently observed for 1-2 days. For the mouse brain glioma model:
(1) A single cell suspension of mouse glioma cells (GL261) in logarithmic growth phase was prepared. The cell concentration was adjusted to 1.0×10⁶ cells/mL using sterile PBS as solvent;
(2) C57BL/6 female mice, 6-8 weeks old, weighing 18-20 g, were anesthetized with sodium phenobarbital (40 mg/kg) in an superclean bench and shaved to facilitate tumor-bearing and tumor measurement;
(3) GL261 cells (0.1 mL of single cell suspension) were subcutaneously implanted into the upper right area of the back of the mouse with a syringe, and an obvious spherical bulge was formed at the injection site;
(4) The mice were returned to the rearing cage and subsequently observed for 10 days. 3.5.2 Grouping and drug treatment of the tumor-bearing mice
(1) When the tumor (about 50 mm³) appeared on the back of the mice, they were randomly divided into groups with 5-6 mice/group (control group and treatment group) and subjected to drugs;
(2) The treatment groups were divided into the group with GSK126 alone (50mg/kg/day), the group with fenofibrate alone (100 mg/kg/day), and the combination group of GSK126 (50 mg/kg/day) with fenofibrate (100 mg/kg/day). Drugs, including GSK126and fenofibrate, were purchased from Shanghai Lanmu Chemical Co., Ltd., and the solvent was formulated according to the instruction, while the control group was treated with the same amount of solvent. The administration frequency was 3 times/4 days, and the length (a) and width (b) of the tumor as well as the changes of the body weight of the mice were recorded with a vernier caliper and a weighing balance every 2 days.
(3) After 14 days of treatment, the mice were sacrificed and the corresponding tissue samples were preserved for downstream experiments.

3.6 Anti-tumor effect of the combination of EZH2 inhibitor with PPARα agonist in a dyslipidemia tumor-bearing mouse model

3.6.1 Establishment of dyslipidemia mouse model
(1) C57BL/6 female mice (body weight of 18-20 g) of 6-8 weeks old were randomly divided into groups, and then the mice in the dyslipidemia group were fed with commercially available “Western Diet” for 2 months to establish a dyslipidemia mouse model.

1(2) Identification of mice with dyslipidemia: the eyeball blood of mice was collected into EP tubes and naturally coagulated at 4°° C. It was then centrifuged at 2000 rpm at 4° C. for 10 min. Serum was collected into clean EP tubes, stored at low temperature and sent to the Department of Laboratory Animal Science, Peking University Health Science Center for biochemical analysis (TC, TG, LDL-C, HDL-C, etc.). 3.6.2 Construction of tumor-bearing mouse model on the basis of the dyslipidemia mouse model
(1) A single cell suspension of tumor cells (MC38) in logarithmic growth phase was prepared. The cell concentration was adjusted to 1.0×10⁷ cells/mL using sterile PBS as solvent.
(2) The dyslipidemic mice prepared in Section 3.5.1 were anesthetized with sodium phenobarbital (40 mg/kg) in an superclean bench and shaved to facilitate tumor-bearing and tumor measurement;
(3) MC38 cells (0.1 mL of single cell suspension) were subcutaneously implanted into the upper right area of the back of the mouse with a syringe, and an obvious spherical bulge was formed at the injection site;
(4) The mice were returned to the rearing cage and subsequently observed for 1-2 days. 3.6.3 Grouping and drug treatment of the tumor-bearing mice with dyslipidemia
(1) When the tumor (about 50 mm3) appeared on the back of the mice, they were randomly divided into groups with 5-6 mice/group (control group and treatment group) and subjected to drugs;
(2) The treatment groups were divided into the group with GSK126 alone (50mg/kg/day), the group with fenofibrate alone (100 mg/kg/day), and the combination group of GSK126 (50 mg/kg/day) with fenofibrate (100 mg/kg/day). Drugs, including GSK126and fenofibrate, were purchased from Shanghai Lanmu Chemical Co., Ltd., and the solvent was formulated according to the instruction, while the control group was treated with the same amount of solvent. The administration frequency was 3 times/4 days, and the length (a) and width (b) of the tumor as well as the changes of the body weight of the mice were recorded with a vernier caliper and a weighing balance every 2 days.
(3) After 14 days of treatment, the mice were sacrificed and the corresponding tissue samples were preserved for downstream experiments.

It will be apparent to those skilled in the art that various modifications and variations can be made in the present invention without departing from the spirit and scope of the invention. Thus, the present invention is intended to include such modifications and variations provided they come within the scope of the appended claims and their equivalents.

1-10. (canceled)

11. A pharmaceutical composition for enhancing the anti-tumor effect of an EZH2inhibitor, wherein the pharmaceutical composition comprises an EZH2 inhibitor and a PPARα agonist, wherein the PPARα agonist enhances the anti-solid tumor effect of the EZH2 inhibitor.

12. The pharmaceutical composition according to claim 11, wherein the mass ratio of the EZH2 inhibitor to the PPARα agonist is from 1:1 to 1:10.

13. The pharmaceutical composition according to claim 11, wherein:
the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer.

14. The pharmaceutical composition according to claim 13, wherein the solid tumor is selected from breast cancer, melanoma, lung cancer, colon cancer, liver cancer, gastric cancer, or brain cancer, and preferably, the brain cancer is selected from meningioma, glioma (e.g., astrocytoma, oligodendroglioma), or medulloblastoma.

15. The pharmaceutical composition according claim 11, wherein:
the EZH2 inhibitor is selected from Tazemetostat (EPZ-6438), GSK126, Lirametostat (CPI-1205), SHR2554 or PF-06821497; the PPARα agonist is selected from fenofibrate, clofibrate, bezafibrate, clinofibrate, ciprofibrate, etofibrate, gemfibrozil, WY-14643 (pirinixic acid), GW-7647(2-(4-(2-(1-1-cyclohexylbutyl)-3-cyclohexylureido)ethyl)phenylthio)-2-methylpropionic acid) or pemafibrate ((R)-2-{3- {[N-(benzoxazol-2-yl)-N-3-(4-methoxyphenoxy)propyl]aminomethyl}phenoxy}butyric acid).

16. The pharmaceutical composition according claim 12, wherein:
the EZH2 inhibitor is selected from Tazemetostat (EPZ-6438), GSK126, Lirametostat (CPI-1205), SHR2554 or PF-06821497; the PPARα agonist is selected from fenofibrate, clofibrate, bezafibrate, clinofibrate, ciprofibrate, etofibrate, gemfibrozil, WY-14643 (pirinixic acid), GW-7647(2-(4-(2-(1-1-cyclohexylbutyl)-3-cyclohexylureido)ethyl)phenylthio)-2-methylpropionic acid) or pemafibrate ((R)-2-{3-{[N-(benzoxazol-2-yl)-N-3-(4-methoxyphenoxy) propyl]aminomethyl}phenoxy}butyric acid).

17. The pharmaceutical composition according claim 13, wherein:
the EZH2 inhibitor is selected from Tazemetostat (EPZ-6438), GSK126, Lirametostat (CPI-1205), SHR2554 or PF-06821497; the PPARα agonist is selected from fenofibrate, clofibrate, bezafibrate, clinofibrate, ciprofibrate, etofibrate, gemfibrozil, WY-14643 (pirinixic acid), GW-7647 (2-(4-(2-(1-1-cyclohexylbutyl)-3-cyclohexylureido)ethyl)phenylthio)-2-methylpropionic acid) or pemafibrate ((R)-2-{3-{[N-(benzoxazol-2-yl)-N-3-(4-methoxyphenoxy) propyl]aminomethyl} phenoxy}butyric acid).

18. The pharmaceutical composition according to claim 11, wherein the EZH2 inhibitor is GSK126 and the PPARα agonist is fenofibrate.

19. The pharmaceutical composition according to claim 12, wherein the EZH2 inhibitor is GSK126 and the PPARα agonist is fenofibrate.

20. The pharmaceutical composition according to claim 13, wherein the EZH2 inhibitor is GSK126 and the PPARα agonist is fenofibrate.

21. The pharmaceutical composition according to claim 15, wherein the EZH2 inhibitor is GSK126 and the PPARα agonist is fenofibrate.

22. A pharmaceutical preparation for enhancing the anti-tumor effect of an EZH2inhibitor, wherein the pharmaceutical preparation is prepared from a therapeutically effective amount of the pharmaceutical composition according to claim 11 and a pharmaceutically acceptable carrier.

23. The pharmaceutical preparation according to claim 22, wherein the pharmaceutical preparation is an oral preparation.

24. The pharmaceutical preparation according to claim 23, wherein the oral preparation is an oral liquid, a tablet, a powder, a capsule or a granule.

25. Use of the pharmaceutical composition according to claim 11.

26. The use according to claim 25, wherein: the tumor is a solid tumor, and the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer, and preferably, the solid tumor is selected from breast cancer, melanoma, lung cancer, colon cancer, liver cancer, gastric cancer, or brain cancer, and more preferably, the brain cancer is selected from meningioma, glioma (e.g., astrocytoma, oligodendroglioma), or medulloblastoma.

27. Use of the pharmaceutical preparation according to claim 22 in preparation of an anti-tumor drug.

28. The use according to claim 27, wherein: the tumor is a solid tumor, and the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer, and preferably, the solid tumor is selected from breast cancer, melanoma, lung cancer, colon cancer, liver cancer, gastric cancer, or brain cancer, and more preferably, the brain cancer is selected from meningioma, glioma (e.g., astrocytoma, oligodendroglioma), or medulloblastoma.

29. Use of a PPARα agonist in the preparation of a drug for enhancing the efficacy of an EZH2 inhibitor against a solid tumor, wherein:
the solid tumor is selected from breast cancer, prostate cancer, melanoma, osteosarcoma, neuroblastoma, pancreatic cancer, lung cancer, rhabdomyosarcoma, Ewing's sarcoma, bladder cancer, colon cancer, liver cancer, ovarian cancer, cervical cancer, nasopharyngeal cancer, laryngeal cancer, gastric cancer, renal cancer, head and neck tumor, esophageal cancer, testicular cancer, thyroid cancer or brain cancer, and preferably, the solid tumor is selected from breast cancer, melanoma, lung cancer, colon cancer, liver cancer, gastric cancer, or brain cancer, and more preferably, the brain cancer is selected from meningioma, glioma (e.g., astrocytoma, oligodendroglioma), or medulloblastoma; the EZH2 inhibitor is selected from Tazemetostat (EPZ-6438), GSK126, Lirametostat (CPI-1205), SHR2554 or PF-06821497; and
the PPARα agonist is selected from fenofibrate, clofibrate, bezafibrate, clinofibrate, ciprofibrate, etofibrate, gemfibrozil, WY-14643 (pirinixic acid), GW-7647 (2-(4-(2-(1-1-cyclohexylbutyl)-3-cyclohexylureido)ethyl) phenylthio)-2-methylpropionic acid) or pemafibrate ((R)-2-{3-{[N-(benzoxazol-2-yl)-N-3-(4-ethoxyphenoxy) propyl]aminomethyl}phenoxy}butyric acid).
